(19) United States
(12) Patent Application Publication
(10) Pub. No.: US 2025/0134725 A1
(43) Pub. Date: May 1, 2025

(54) SKIN CARE FOR EXTREMELY LOW BIRTHWEIGHT INFANTS

(71) Applicant: Children's Hospital Medical Center, Cincinnati, OH (US)

(72) Inventor: Katherine Elizabeth McKeown, Florence, KY (US)

(21) Appl. No.: 18/934,187

(22) Filed: Oct. 31, 2024

Related U.S. Application Data
(60) Provisional application No. 63/594,793, filed on Oct. 31, 2023.

(51) Int. Cl. A61F 13/14; A61F 13/00; A61F 13/02; A61F 13/0246; A61L 15/44; A61L 15/58

(52) U.S. Cl. CPC A61F 13/148; A61F 13/00063; A61F 13/0253; A61F 13/0266; A61L 15/44; A61L 15/58; A61F 2013/00182; A61F 2013/00251; A61F 2013/0091; A61F 2013/00953; A61L 2300/204; A61L 2300/206; A61L 2430/34

(57) ABSTRACT

A securement device for an infant including a lower layer having a silicone adhesive and a center opening configured to receive an umbilical cord of the infant. An upwardly extending first securement tab and an upwardly extending second securement tab are positioned on opposing sides of the center opening. The first securement tab and the second securement tab define a bridge capturing an umbilical line. The lower layer includes a first antimicrobial, and the first securement tab includes a second antimicrobial.

### CROSS-REFERENCE TO RELATED APPLICATION

The present application claims priority to U.S. Provisional Patent Application Ser. No. 63/594,793, filed Oct. 31, 2023, the disclosure of which is expressly incorporated by reference.

### BACKGROUND AND SUMMARY OF THE DISCLOSURE

The present invention relates generally to skin care and, more particularly, to a system and method for extremely low birthweight (ELBW) infant skin wound management.

While the survival rate for extremely low birthweight (ELBW) infants has increased significantly in recent years, prematurity, infections, and birth defects remain leading causes of neonatal mortality. The innate immune function of the skin is underdeveloped in periviable infants as they lack a competent epidermal barrier. Globally, clinicians call for caution and careful consideration of skin care practices, particularly for periviable infants. Published evidence is extremely limited, as the youngest infants are frequently excluded from randomized controlled trials. Skin injuries in NICU patients, including epidermal stripping, pressure injury, extravasation injury, and diaper dermatitis, have been described for many years. Skin injuries continue to occur despite instituting validated quality improvement methods which have shown to improve outcomes. The need for continued improvement in skin care devices and related methods of care are evident.

The skin care devices and related methods of the present disclosure are motivated by consideration of how the varied skin care practices and topical products used in their care might impact the maturation of the preterm skin. The skin challenges presented by periviable infants need to revitalize the focus on specific product development designed specifically for ELBW infants.

Wounds heal differently in neonates and adults. Neonatal skin can generate granulation tissue, elastin, collagen and fibroblasts more rapidly than adults. Granulation tissue and extracellular matrix develop more quickly to facilitate wound closure. Wound healing occurs in stages: (1) hemostasis, (2) inflammation, (3) proliferation, and (4) remodeling/modulation. After wounding, hemostasis occurs as blood vessels vasoconstrict and platelets interact with extracellular matrix components, including fibronectin and collagen, to stick to the vessel wall. Fibronectin, fibrin, thrombospondin and vitronectin form a clot to prevent bleeding. Endothelial and smooth muscles cells orchestrate to restore damaged blood vessels. In the inflammatory stage, the damaged tissues signal immune cells, i.e., macrophages, T cells, Langerhans cells and mast cells, to activate inflammation and production of cytokines and chemokines. They attract neutrophils, leucocytes and monocytes to the wound and generate toxins to destroy infectious agents, produce cytokines (IL-1, IL-6, TNFα) and chemokines and debride necrotic tissue. Finally, macrophages engulf bacteria and clear neutrophils, signaling completion of the inflammatory phase.

In the proliferative stage, keratinocytes, fibroblasts, macrophages and endothelial cells interact in an orchestrated process wherein keratinocytes generate metalloproteinases and produce extracellular matrix proteins (ECM) to form new basement membrane. Keratinocytes move from opposite sides of the wound to coalesce and form new epidermal layers from bottom to top of the wound. Stem cells from skin elements, e.g., hair follicles, sebaceous glands, respond to injury and contribute stem cells to the repair process. Growth factors (e.g., TGFβ) activate fibroblasts to produce collagen and ECM necessary for dermal repair. Finally, remodeling of tissue occurs via changes in the ECM and fibroblasts. The clot that formed to halt bleeding is “substituted” with fibronectin, proteoglycans and hyaluronan to create the complex collagen fibrils.

With reference to FIGS. 1A and 1B, skin wounds are often classified by depth as (1) superficial, (2) partial thickness and (3) full thickness, i.e., based on the depth of the injury. Superficial wounds 2 have damage to the epidermis 4 of the skin. They can vary in severity, ranging from loss of some or all of the stratum corneum (SC) to complete loss including the basal layer. Partial thickness wounds include the epidermis 4 and some or all of the dermis 5. The epidermis 4 is typically the top one to three layers of skin, while the dermis 5 is typically several layers of skin below the epidermis 4. A connecting membrane 6 typically connects the epidermis 4 to the dermis 5 via an adhesion tension. Full thickness injuries 7 extend into the subcutaneous tissue 8 and may include the fascia and muscle, particularly in ELBW infants where subcutaneous tissue is absent. Superficial and partial thickness wound healing is shown schematically in FIGS. 1A and 1B, respectively.

Two strategies for ELBW infant skin wound management are recommended, namely moist care and dry care. The literature acknowledges the difficulty in determining the “optimum” moisture/dryness to be achieved for wound healing. In general, provision of moisture to the wound is necessary, as moisture enables cell migration, cell signaling, re-epithelialization, robust extracellular matrix, formation, etc., to increase healing rate and reduce scar formation. The unaffected skin surrounding a wound can become damaged (macerated) if the wound treatment inadvertently increases the moisture in those areas. Dressings play an important role in managing moisture to create the “optimum” moisture, i.e., not too wet and not too dry. Clinicians have recommended moderation in ELBW wound treatment, including dry treatment. Addition of moisture may risk maceration and a delay in barrier formation in a humid incubator environment. Low hydration and dry, scaly skin is commonly observed in neonates following the transition from wet to relatively dry conditions at birth before increasing during weeks 2-4. The hydration decrease and appearance of scale is somewhat delayed in premature infants. As the premature infant skin barrier rapidly matures, epidermal proteins inhibit the enzymes that cause the outermost layers to desquamate. Reduced desquamation is protective for premature infants as maintenance of that layer helps reduce water loss.

The specific properties of dressings, including relative occlusivity, measured as the water vapor transport rate (moisture weight per time) and the capacity to absorb fluid must be carefully considered for ELBW skin care. Occlusive dressings, namely those with relatively low water vapor transport rates, are known to facilitate formation of granulation tissue and are necessary for healing partial and full thickness wounds. In contract, dressings that were not occlusive, namely semipermeable with relatively high water vapor transport, promoted re-epithelization needed for superficial wounds and for the later stages of partial and full thickness wounds. Unfortunately, technical properties, such as water vapor transport rates, have been reported for only a few commercially available dressings.

According to an illustrative embodiment of the present disclosure, a skin care device for an infant includes a base having an upper surface, a lower surface and a center opening configured to receive an umbilical cord of the infant. The lower layer includes a silicone adhesive. The silicone adhesive is secured to an outer epidermis layer of the infant, and the outer epidermis layer coupled to an inner dermis layer of the infant. The adhesive tension between the silicone adhesive and the outer dermis layer is less than the adhesive tension between the outer epidermis layer and the inner dermis layer. A liner is removably coupled to the silicone adhesive. An upper layer is coupled to the upper surface of the lower layer.

According to another illustrative embodiment of the present disclosure, a skin care device for an infant includes a lower layer having an upper surface, a lower surface, a center opening configured to receive an umbilical cord of the infant, and a positioning slot extending outwardly from the center opening. The lower surface includes an adhesive. An upwardly extending first securement tab and an upwardly extending second securement tab are positioned on opposing sides of the center opening. The lower layer includes a first antimicrobial, and at least one of the first securement tab and the second securement tab includes a second antimicrobial.

According to a further illustrative embodiment of the present disclosure, a skin care device for an infant includes a lower layer having an upper surface, a lower surface and a center opening configured to receive an umbilical cord of the infant. The lower surface includes an adhesive. A positioning slot extends within the lower layer outwardly from the center opening. The lower layer includes a sensor opening receiving a temperature probe. A liner is removably coupled to the adhesive of the lower surface. An upwardly extending first securement tab and an upwardly extending second securement tab are positioned on opposing sides of the center opening. At least one of the first securement tab and the second securement tab includes an adhesive facing the other of the second securement tab and the first securement tab. The first securement tab is coupled to the second securement tab to define a first bridge extending over the center opening and capturing a first umbilical line between the first securement tab and the second securement tab. The lower layer is semi-transparent for phototherapy transmissions of up to 490 nm. The lower layer includes a water vapor transmission rate of at least 2000 g/m² per 24 hours.

Additional features and advantages of the present invention will become apparent to those skilled in the art upon consideration of the following detailed description of the illustrative embodiment exemplifying the best mode of carrying out the invention as presently perceived.

### BRIEF DESCRIPTION OF THE DRAWINGS

The foregoing aspects and many of the intended advantages of this invention will become more readily appreciated as the same becomes better understood by reference to the following detailed description of exemplary embodiments when taken in conjunction with the accompanying drawings, wherein:

FIG. 1A illustrates processes in healing of superficial wounds;

FIG. 1B illustrates processes in healing of partial thickness wounds;

FIG. 2 is a front perspective view of an illustrative securement device of the present disclosure, shown in a fully deployed mode and secured to an infant;

FIG. 3 is an upper perspective view of the illustrative securement device of FIG. 2, shown in an undeployed mode with the paper liners of the lower layer removed for clarity;

FIG. 4 is a lower perspective view of the illustrative securement device of FIG. 3, shown in an undeployed mode with the paper liners of the lower layer removed for clarity;

FIG. 5 is a cross-sectional view of the illustrative securement device of FIG. 3 taken along line 5-5;

FIG. 6 is a detail view of FIG. 5;

FIG. 7 is a lower perspective view of the illustrative securement device of FIG. 2, showing an initial installation step on an infant;

FIG. 8 is an upper perspective view of the illustrative securement device of FIG. 7, showing a further installation step around an umbilical cord of the infant;

FIG. 9 is an upper perspective view of the illustrative securement device of FIG. 8, showing a further installation step around an umbilical cord of the infant with the lower layer secured to the skin of the infant;

FIG. 10 is an upper perspective view of the illustrative securement device of FIG. 9, showing a further installation step around an umbilical cord of the infant with first and second umbilical lines coupled to the umbilical cord;

FIG. 11 is an upper perspective view of the illustrative securement device of FIG. 9, showing a further installation step around an umbilical cord of the infant with first and second securement tabs extending upwardly from the lower layer;

FIG. 12 is an upper perspective view of the illustrative securement device of FIG. 9, showing a further installation step around an umbilical cord of the infant with first and second securement tabs coupled together to define a first securement bridge securing the first umbilical line;

FIG. 13 is an upper perspective view of the illustrative securement device of FIG. 9, showing a further installation step around an umbilical cord of the infant with third and fourth securement tabs extending upwardly from the lower layer;

FIG. 14 is an upper perspective view of the illustrative securement device of FIG. 13, showing a further installation step around an umbilical cord of the infant with third and fourth securement tabs coupled together to define a second securement bridge securing the second umbilical line; and

FIG. 15 is a cross-sectional view of the illustrative first securement bridge taken along line 15-15 of FIG. 14.

### DETAILED DESCRIPTION OF THE DRAWINGS

The embodiments of the invention described herein are not intended to be exhaustive or to limit the invention to the precise forms disclosed. Rather, the embodiments selected for description have been chosen to enable one skilled in the art to practice the invention.

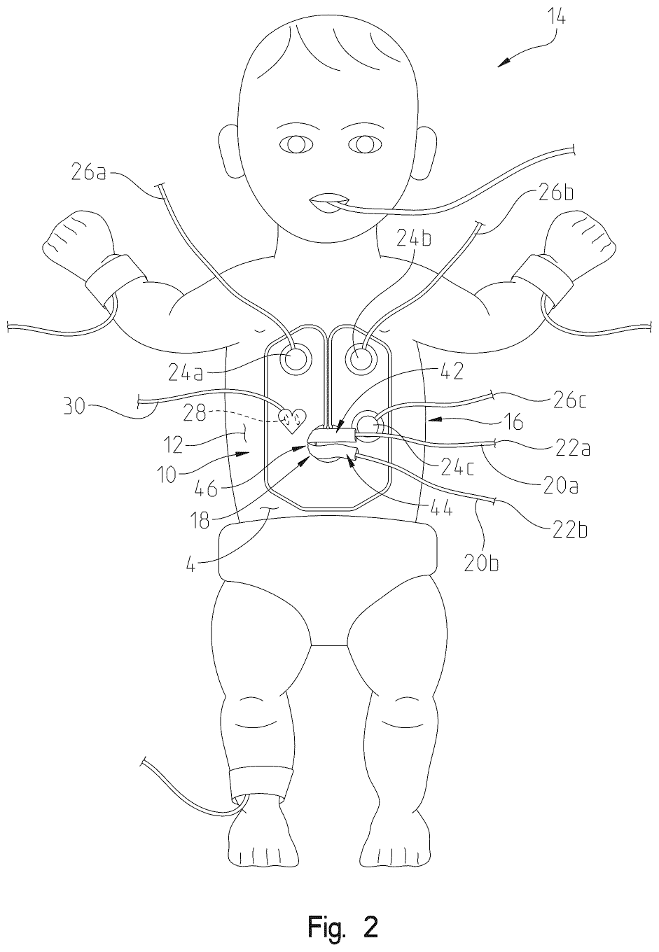

With reference to FIG. 2, an illustrative skin care or securement device 10 is shown secured to the epidermis layer 4 of skin 12 of an infant 14 in an active or deployed mode. While the illustrative skin care device 10 is shown for use on a neonate (less than 28 days in age), it should be appreciated that it has applicability with a variety of patients of different ages.

With further reference to FIG. 2, the illustrative skin care device 10 is shown secured to an abdomen 16 of the infant 14 around an umbilical cord 18. Umbilical lines 20a, 20b are illustratively secured in position by the securement device 10. As further detailed herein, the skin care device 10 may be secured to the skin 12 of the infant 14 in other locations to provide desired treatment.

The umbilical lines 20a, 20b illustratively include an umbilical arterial line or umbilical artery catheter (UAC) 20a, and an umbilical ventricular line or umbilical venous catheter (UVC) 20b. The umbilical arterial line 20a may be of conventional design as including a flexible tube 22a that provides access to the arterial system of the infant 14. The umbilical arterial line 20a may be used to monitor the baby's blood pressure and blood gases, and to provide access for medical treatment. The umbilical ventricular line 20b may be of conventional design as including a flexible tube 22b for providing fluids and/or medicines to the infant 14.

Illustratively, the skin care device 10 supports a plurality of different biometric sensors. For example, electrocardiogram (ECG) contacts or probes 24a, 24b, 24c may be attached to the skin care device 10 and electrically coupled to an electrocardiogram machine (not shown) by lead wires 26a, 26b, 26c to measure electrical activity of the infant's heart. Additionally, a temperature sensor or probe 28 may be coupled to the skin care device 10 and is electrically coupled to a processor (not shown) by a lead wire 30 to measure the infant's body temperature. In other illustrative embodiments, the biometric sensors 24 and 28 may be in wireless communication with respective processors.

In certain other illustrative embodiments, other biometric sensors may be coupled to the skin care device 10, for example a pulse oximeter (not shown) to measure the amount of oxygen in the blood of the infant 14. In an illustrative embodiment, a single combined sensor may provide readings wirelessly of an ECG, a temperature sensor and/or a pulse oximeter.

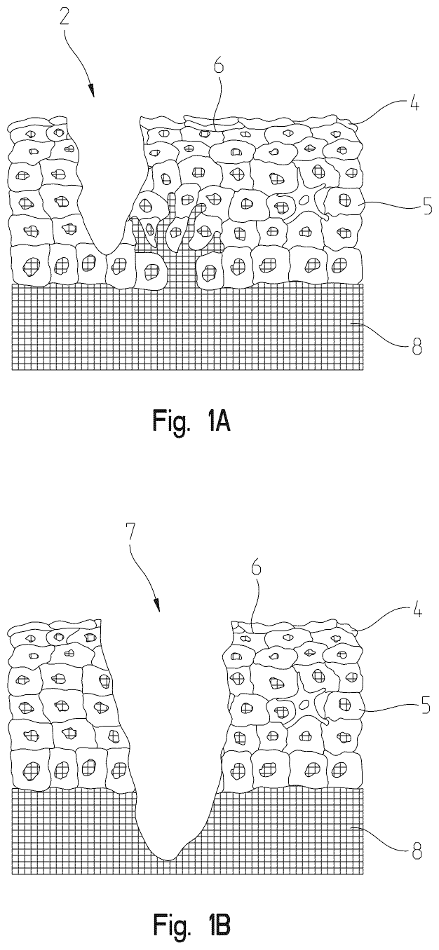

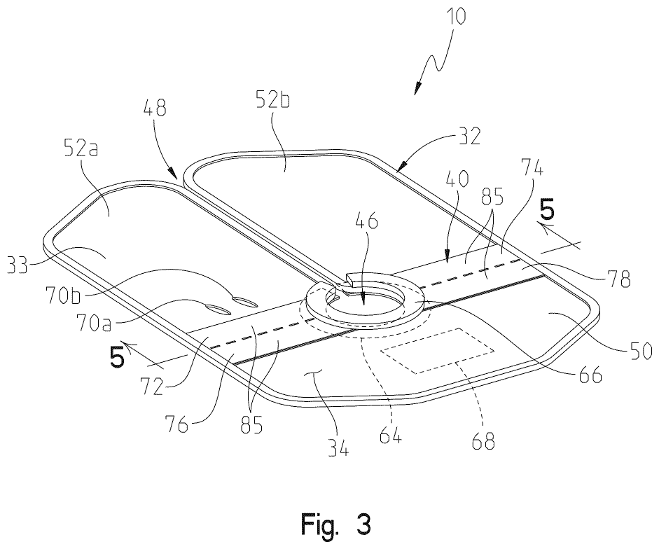

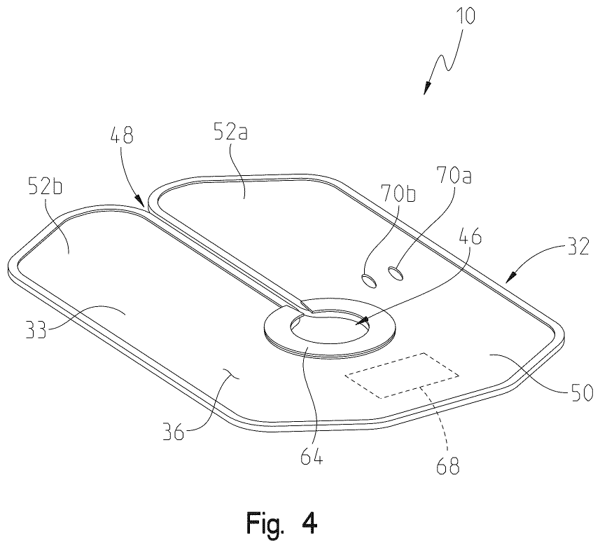

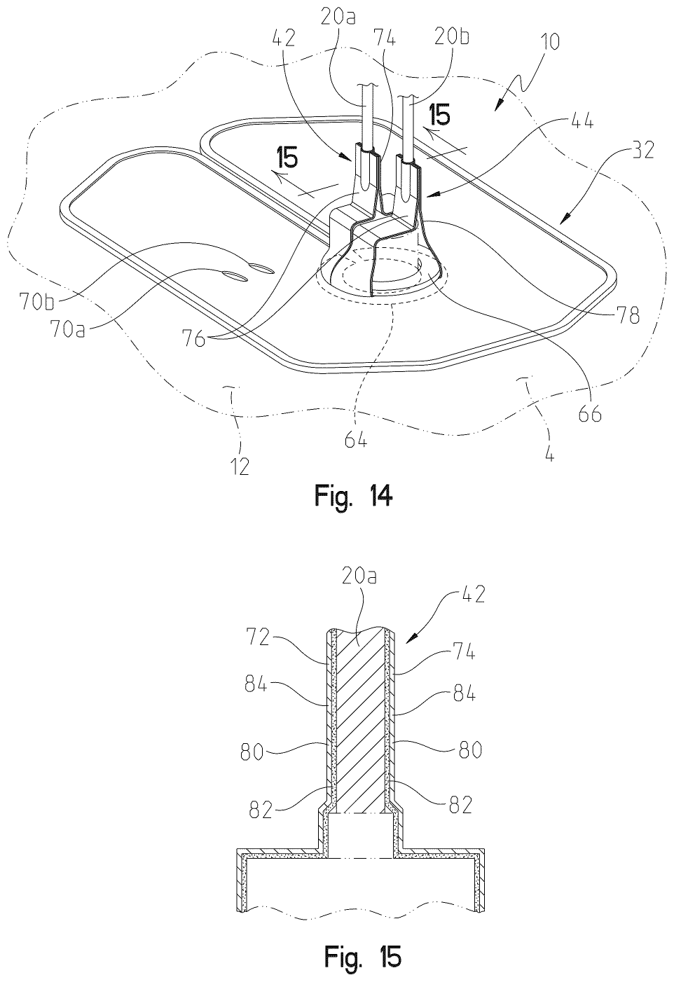

The skin care device 10 illustratively includes a lower layer or base 32 having a non-woven substrate 33 with an upper surface 34 and a lower surface 36. The skin care device 10 in FIGS. 3-6 is shown in a stored or undeployed mode with releasable paper liners of the base 32 (detailed below) removed for clarity. An upper layer 40 is coupled to the base 32 and in the undeployed mode extends parallel to the base 32. In the deployed mode, the upper layer 40 defines first and second securement bridges 42 and 44, as further defined herein (FIGS. 1 and 14).

The non-woven substrate 33 illustratively comprises a polyurethane membrane which provides an occlusive dressing defining a protective barrier to external contamination. More particularly, the substrate 33 is semi-permeable and highly porous for both water vapor and oxygen transport, but is impermeable to micro-organisms from outside the base 32.

In order to allow for maturation of the skin underneath the base 32, the substrate 33 includes a water vapor transmission rate (WVTR) of at least 2000 grams per meter squared (g/m²) per 24 hours. For neonates, due to the relative lack of development of their skin, the WVTR is illustratively between 2000 g/m² per 24 hours and 3800 g/m² per 24 hours. The upper end of the WVTR range may be greater for more mature patients due to further development of their skin (in other words, based upon the gestational age of the baby 14).

Further illustratively, the substrate 33 of the base 32 is semi-transparent for phototherapy transmissions at no less than 30 mW/cm2 per nm (or up to 490 nm). The substrate 33 transparency of at least 490 nm is the level of irradiance for intensive phototherapy, which is a treatment for hyperbilirubinemia in infants. This level of transparency also facilitates the reading of ECG probes 24 through the base 32. In addition, the base 32 illustratively has a pH of 5.5 to maintain a proper acid mantle balance that is known to reduce infection in neonates.

A center opening 46 illustratively extends through the substrate 33 and is configured to receive the umbilical cord 18. A positioning slot 48 extends within the base 32 outwardly from the center opening 46. As such, the base 32 is divided into a bottom portion 50, and first and second upper portions 52a and 52b separated by the positioning slot 48.

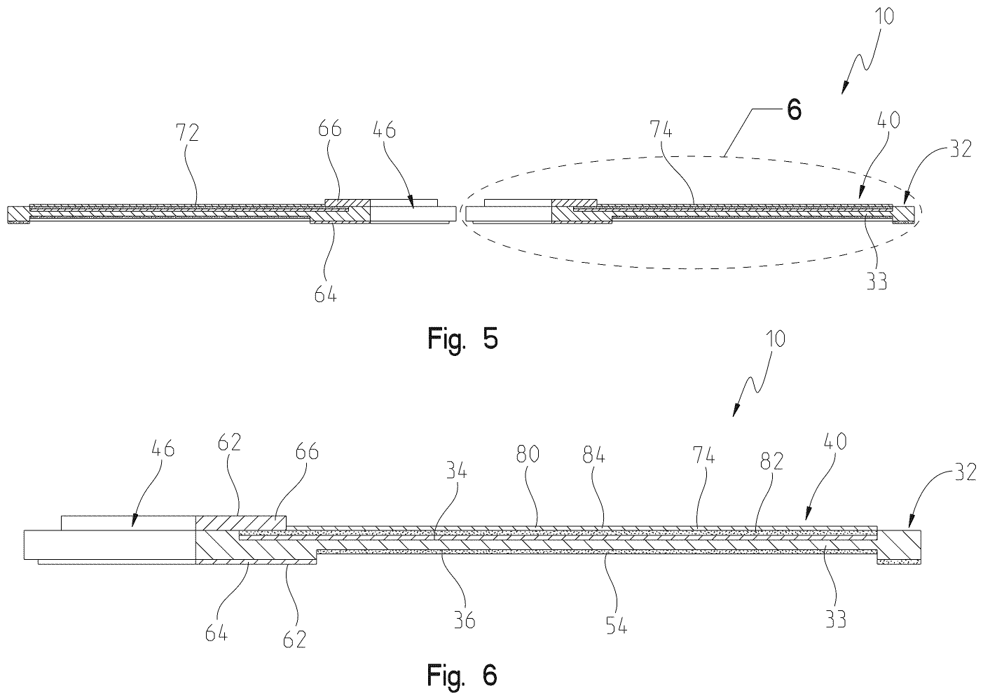

With reference to FIGS. 5 and 6, a skin friendly adhesive 54, illustratively a silicone adhesive, is illustratively supported by the lower surface 36 of the base 32. More particularly, the adhesive 54 permits the base 32 to be removable and reseatable on the skin 12 of the infant 14. The adhesive 54 is secured to the outer epidermis 4 of the infant 14. As detailed above, the outer epidermis 4 is coupled to the inner dermis 5 of the infant 14 by adhesive tension via a connecting membrane 6. In order to be skin friendly, the adhesive 54 has a low adhesion force or tension. More particularly, adhesive tension between the adhesive 54 of the base 32 and the outer epidermis 4 of the skin 12 is less than the adhesive tension between the outer epidermis 4 and the inner dermis 5 of the skin 12 in order to prevent layers of the outer epidermis 4 from being removed when the skin care device 10 is uncoupled from the skin 12 of the infant 14.

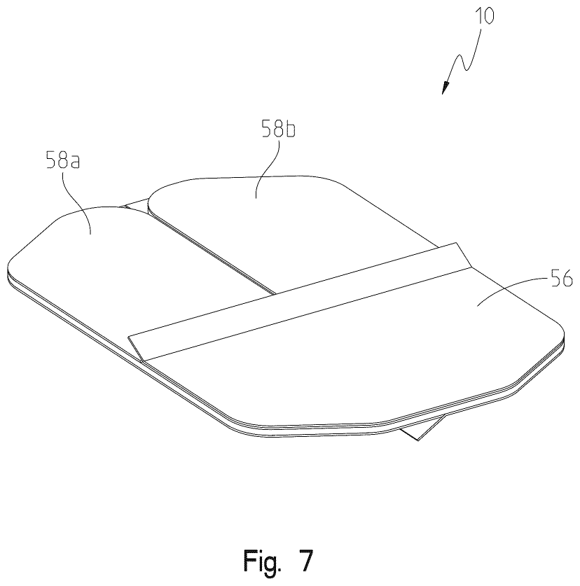

Lower paper liners 56, 58a, 58b are releasably secured for the lower surface 36 at the bottom portion 50, and the upper portions 52a and 52b (FIG. 7). Upper paper liners 60a, 60b are releasably secured to the upper surface 34 of the base 30. The paper liners 56, 58a, 58b, 60a, 60b are removably coupled to the substrate 33 of the base 32 during transport and/or storage of the skin care device 10.

With further reference to FIGS. 5 and 6, a first antimicrobial 62 is illustratively supported by the lower surface 36 of the base 30. Illustratively, the first antimicrobial 62 is skin friendly and may be a separate layer or impregnated into the base 30. For example, the antimicrobial 62 may be impregnated into a lower center layer 64 and/or an upper center layer 66. The center layers 64 and 66 may be arcuate rings of a mesh substrate extending around the center opening 46. In one illustrative embodiment, the first antimicrobial 62 comprises dialkylcarbamoyl chloride (DACC). In certain illustrative embodiments, the lower layer 64 may include a skin friendly antimicrobial 62, while the upper layer 66 may include a different antimicrobial, such as chlorhexidine (CHG). The first antimicrobial 62 (DACC) does not tend to burn skin 12, while the second antimicrobial (CHG) tends to irate and/or burn skin 12. Illustratively, the first antimicrobial 84 is configured to reduce the bacterial load at and adjacent to the umbilical cord 18, including on the skin 12 of the infant 14.

Illustratively, the base 32 may be fenestrated. For example, the substrate 33 of the base 32 may include a transparent window 68 thereby providing visual access to the skin 12 of the infant 14. In other illustrative embodiments, multiple windows 68 may be provided, or the substrate 33 itself may be transparent. First and second openings 70a, 70b may be formed within the substrate 33 of the base 32, and are configured to receive the temperature probe 28. Multiple openings 70a, 70b provide flexibility in positioning of the temperature probe 28.

The upper layer 40 includes a first securement tab 72 and a second securement tab 74. In the undeployed mode, the first securement tab 72 and the second securement tab 74 extends outwardly from the opening 46 parallel to the base 30. In the deployed mode, the first securement tab 72 and the second securement tab 74 extend upwardly on opposite sides of the center opening 46. Similarly, the upper layer 40 may include a third securement tab 76 and a fourth securement tab 78. In the undeployed mode, the third securement tab 76 and the fourth securement tab 78 extends outwardly from the opening 46 parallel to the base 30. In the deployed mode, the third securement tab 76 and the fourth securement tab 78 extend upwardly on opposite sides of the center opening 46. In the undeployed mode, the first securement tab 72 and the third securement tab 76 may be separated by a perforation, and similarly the second securement tab 74 and the fourth securement tab 78 may be separated by a perforation.

The first and second securement tabs 72 and 74 are coupled together to define the first securement bridge 42 capturing the first umbilical line 20a. The third and fourth securement tabs 76 and 78 define the second securement bridge 44 capturing the second umbilical line 20b. The first securement bridge 42 is spaced apart from the second securement bridge 44, thereby allowing independent manipulation and/or replacement of the umbilical lines 20a, 20b. Sutures (not shown) may be used to help secure the lines 20a, 20b within the securement bridges 42, 44.

With reference to FIGS. 5, 6 and 15, each securement tab 72, 74, 76, 78 may include a substrate 80 supporting an adhesive 82. In one illustrative embodiment, each securement tab 72, 74, 76, 78 may be formed of a self-adhesive polyurethane film (such as Tegaderm). The substrate 80 and adhesive 82 of each securement tab 72, 74, 76, 78 may be transparent so that the user can view the respective umbilical lines 20a, 20b to ensure proper placement. In certain illustrative embodiments, the umbilical lines 20a, 20b include markings visible through the transparent securement tabs 72, 74, 76, 78 so that the caregiver can ensure that the lines 20a, 20b have not moved or become dislodged. While the entire tab 72, 74, 76, 78 may be transparent, in other illustrative embodiments, one or more transparent windows may be formed within the substrates 80.

Illustratively, a second antimicrobial 84 is applied to each tab 72, 74, 76, 78. Illustratively, the second antimicrobial 84 comprises chlorhexidine (CHG) which may be embedded in the substrate 80 or define a separate layer. Illustratively, the second antimicrobial 84 is configured to reduce the bacterial load at and adjacent to the umbilical cord 18, including at the umbilical lines 20a, 20b. The CHG impregnated tabs 72, 74, 76, 78 also facilitate desiccation of the umbilical cord 18.

A releasable paper liner 85 may be applied to the adhesive 82 of each tab 72, 74, 76, 78. The paper liners 85 are removably coupled to the substrate 80 of each tab 72, 74, 76, 78 during transport and/or storage of the skin care device 10.

With reference now to FIGS. 7-15, an illustrative method of applying or installing the skin care device 10 will be described in further detail. The procedure begins by carefully cleansing and blotting dry the skin 12 of the infant 14 at her abdomen and chest with conventional mild, skin friendly cleansers. As shown in FIG. 7, initially the securement device 10 is placed with its opening 36 aligned with the umbilical cord 18. The positioning slot 48 facilitates positioning of the base 32 to wrap around the umbilical cord 18

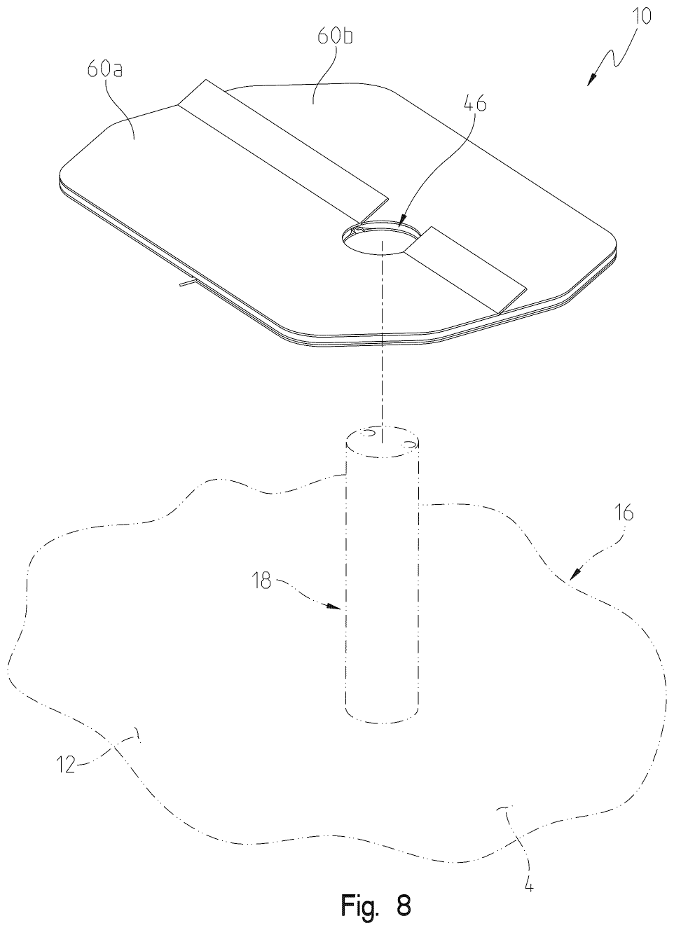

As the base 32 is being properly aligned, the backing paper liners 56, 58a, 58b on the lower surface of the base 32 are then removed exposing the silicone adhesive 54. The silicone adhesive 54 is then applied to the epidermis 4 of the infant's skin 12. With reference to FIG. 8, the upper paper liners 60a, 60b are next removed from the upper surface of base 32 to expose the substrate which will remain in place to act as barrier to germs on the abdomen from, for example, contaminating the umbilical lines 20a, 20b.

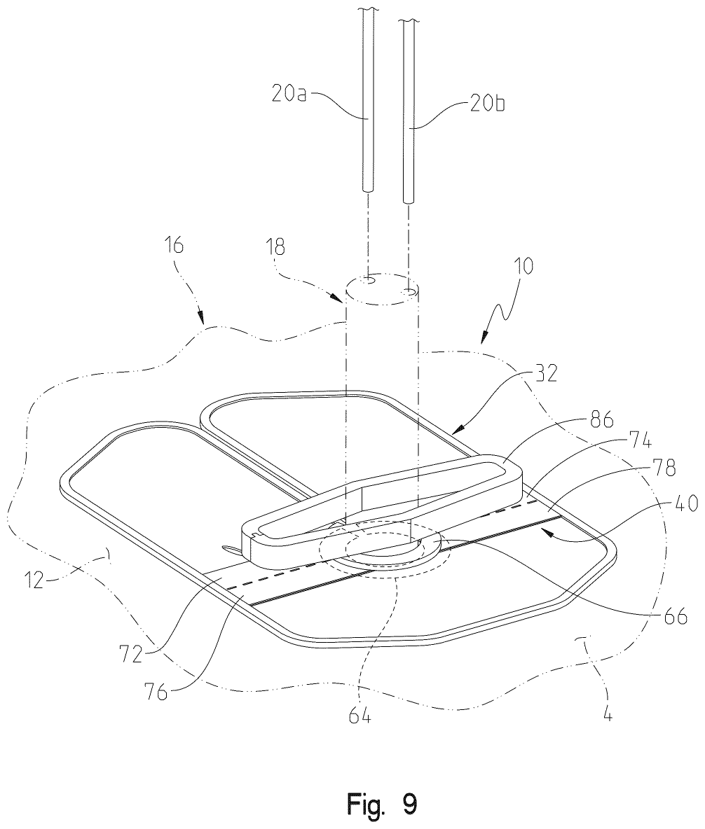

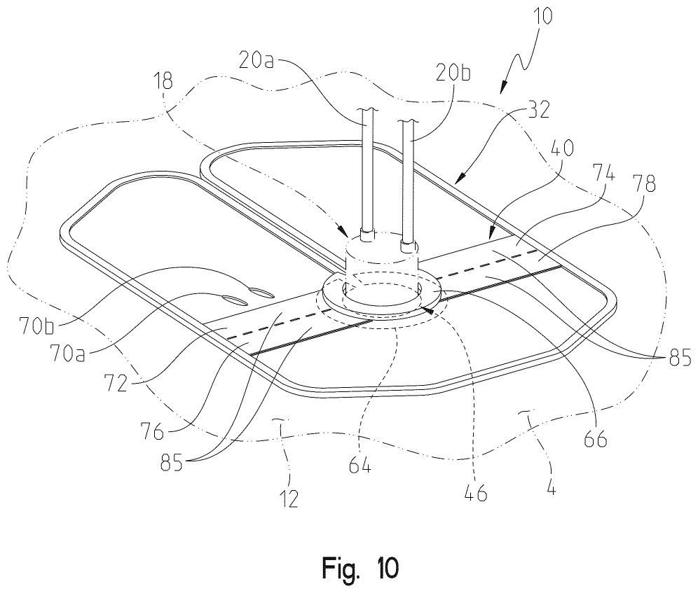

As shown in FIG. 9, umbilical lines 20a, 20b are then aligned with the umbilical cord 18 and in FIG. 10 inserted therein. The umbilical cord 18 is illustratively cut at FIG. 10 and may be clamped using a conventional clamp 86. The clamp 86 may be removed and/or replaced as necessary.

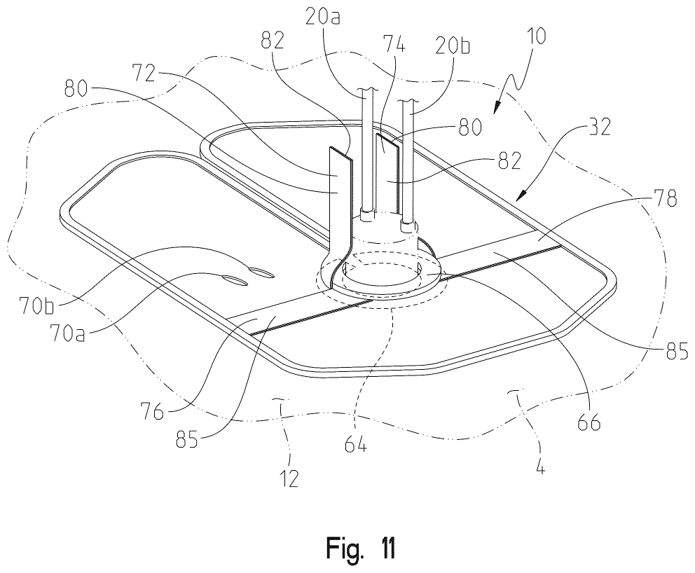

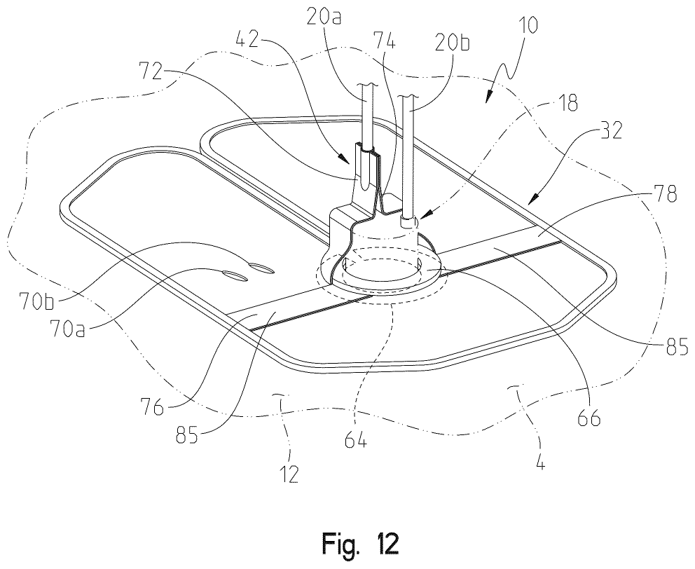

Next, as shown in FIGS. 11 and 12, the first securement tab 72 and the second securement tab 74 are raised up from the parallel position with the base 32. The respective paper backing liners 85 are then removed from each securement tab 72 and 74, thereby exposing the respective adhesive 82. The adhesives 82 on each securement tab 72 and 74 are then brought together and attached to the umbilical line 20a. The umbilical line 20a is captured therebetween by the defined first securement bridge 42. Sutures (not shown) may be used to help secure the line 20a within the securement bridge 42. If sutures are used, then an additional substrate 80 may be secured over the suture holes.

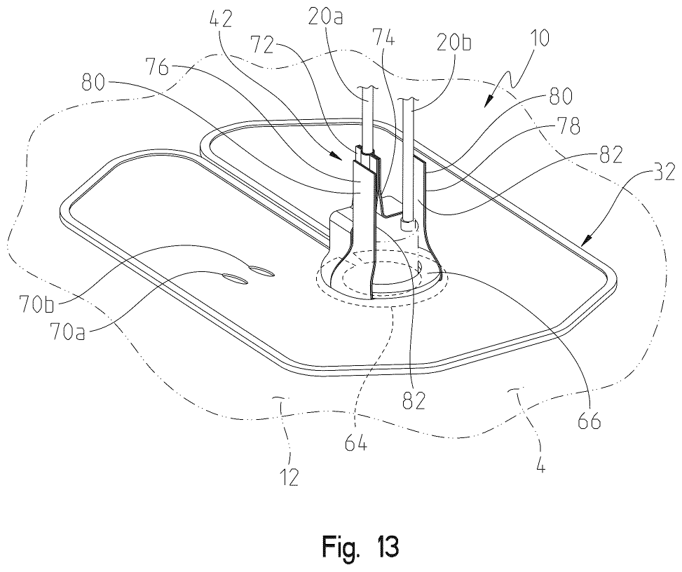

As shown in FIGS. 13 and 14, a similar approach is taken with the third and fourth securement tabs 76 and 78. More particularly, the third and fourth securement tabs 76 and 78 are raised upwardly with their respective backing liners 85 removed and adhered around the second umbilical line 20b. More particularly, the adhesives 82 on each securement tab 76 and 78 are then brought together and attached to the umbilical line 20b. The umbilical line 20b is captured therebetween by the defined second securement bridge 44. Sutures (not shown) may be used to help secure the line 20b within the securement bridge 44. If sutures are used, then an additional substrate 80 may be secured over the suture holes.

Next, the temperature probe 28 may be received in one of the openings 70a, 70b and secured in place with conventional adhesive tape, as desired to sense the temperature of the skin 12. Finally, the ECG electrodes 24a, 24b, 24c may be affixed to the substrate 33 via conventional adhesive tape. It may be appreciated, the probes 28, 24a, 24b, 24c may be removed from the base 32 without damage to the skin 12 of the infant 12.

The skin care device 10 may have different sizes, so they could be changed as needed and with growth. Once the infant 14 is larger and less at risk for infection with more mature skin, they could be allowed to have exposed areas to populate their flora and colonize as usual. The skin care device 10 would enable caregivers to tend to the infant 14 without hands, stethoscope, etc. ripping gelatinous skin, and it would enable families to still do kangaroo care, and slowly establish colonization through the exposed face and small open areas on the dressing rather than the entire infant at one time.

ECG monitoring and temperature sensors could be embedded into the base 32 of the skin care device 10 for placement on the abdomen 16 as is usual practice. Additionally, the base 32 may be radiopaque for x-rays.

If openings 70a, 70b are placed for temperature probe placement and current ECG monitor stickers are placed on the base 32, then these could be removed as usual leaving the skin care device 10 on the infant's skin 12.

The infant 14 would be admitted as usual and once lines are placed and infant 14 is stable, they would be carefully irrigated to cleanse off any potential contaminants, they would be patted lightly dry and the skin care device 10 would be applied carefully. This would decrease the need for high humidity which leads to moisture associated dermatitis. The leads and monitors would have the silicone adhesive as opposed to the currently available hydrogel leads that on a wet infant may cause burn like areas, related to them not being made for a 22-week infant skin maturation.

As noted above, the base 32 of the skin care device 10 is illustratively transparent for continuous assessment. If the areas that monitor the infant 14 are not able to be made transparent, they may be able to be repositionable separately from other portions of the skin care device 10. In addition, the base 32 illustratively has a pH of 5.5 to maintain a proper acid mantle balance that is known to reduce infection in neonates.

Once the infant torso is cleansed and patted dry, the skin care device 10 would be placed by the caregiver surrounding the held up umbilical cord and the skin care device 10 would act as the sterile field for the umbilical line insertion procedure.

The caregiver then scrubs the cord 18 and could extend the disinfectant onto the releasable paper liner. This allows the caregiver to follow their usual procedure in placing lines yet not cause damage. (The infant who would normally be prepped with chlorhexidine gluconate (CHG) or Betadine all over their abdomen is less stimulated by the warm gentle washing of PH balanced baby wash and water and will not feel the cold of the anesthetic preparations related to being covered by the dressing.)

Once the skin care device 10 is placed the caregiver will put the sterile tie or clamp around the cord 18 as usual and measure where to cut the cord from the dressing to the stump with the provided sterile measuring tape. The skin care device 10 will be manufactured by size appropriate to the weight of the baby 14.

The dialkylcarbamoyl chloride (DACC) embedded layer 64 will contact the wharton's jelly once sandwiched to affix the lines 20a, 20b for securing and will be the proper length to the cut on the cord 18. DACC has been proven to decrease the bioburden of wounds and specifically umbilical cords.

Above the DACC, a CHG disc with two openings to accommodate up to 2 5FR catheters as well as their sutures may be positioned.

Finally, the securement tabs 72, 74, 76, 78 of the skin care device 10 will illustratively house these catheters 20a, 20b and sutures. These may be made of clear thick but soft plastic (e.g., polyamide mesh), but include regular adhesive that will also be coated with CHG to keep germs off of the lines 20a, 20b and sutures.

Each side of the securement tabs 72, 74, 76, 78 of the skin care device 10 may have enough border to adhere to the other side which will be identical and be able to come together to form an occlusive dressing to house wharton's jelly and the lines 20a, 20b and whatever umbilical skin is negligibly exposed. If that skin is compromised from the initial scrub, the DACC coated part of the skin care device 10 will allow for healing and wicking away of cleanser and bacteria for a complete healing process.

The securement tabs 72, 74, 76, 78 of the skin care device 10 may include perforations to be able to discontinue one line at a time while continuing the securement of the opposite line.

In conclusion, high rates among neonates of hospital acquired infection with coagulase-negative staphylococci (>49%) have been shown to be able to be reduced by introducing standardized central catheter care protocols. The impact of checklists including guidelines on how to secure and cleanse have proven successful in lowering the rates of central line associated blood stream infection rates which could dramatically impact survival rates of the neonatal population. Biofilms from skin breakdown are breading grounds for opportunistic infections. Central venous line colonization is a major risk factor for the development of such infections. Bloodstream infections associated with central venous catheter insertion are a significant cause of morbidity, disinfection of the catheter insertion site with an antiseptic solution before catheter insertion and during follow-up care is essential to prevent local catheter infection, and transparent dressings are an advantage to prevent any unnecessary exposure of central venous sites. There is a reduction in complications associated with central venous catheter infections with strictly adhered to policies and dressings that provide occlusivity.

Known procedures and products typically do not address the following issues that the illustrative skin care device 10 of the present disclosure is configured to address:
1. Conventional devices lack a barrier that extends over the entire neonatal abdomen that is proven to decrease bacteria to the site after the placement of the lines, silicone dressings provide this assistance.
2. Conventional methods to secure lines have adhesives that lead to breakdown and are used on top of denuded skin burned from antiseptic use and from mechanical damage from scrubbing the skin.
3. Conventional devices do not impregnate DACC dressings which are shown to reduce central catheter and wound infections when placed at the insertion site and on compromised skin and wounds while enabling healing and decreasing the bioburden on those tissues including the umbilical cord.
4. Conventional devices do not target CHG to the site of the line rather than to the skin, which is where the neonate cannot use these devices related to the absorption of noxious chlorhexidine and its neurotoxic effects to neonates related to absorption.
5. Conventional devices do not fold flat to allow for prone positioning as well as kangaroo care while the umbilical line is safely secured and occlusive to the outside environment.
6. Current methods fail to keep all strong adhesives off of the abdomen while allowing for the line and insertion site to be occluded from outside bacteria.

While the above-identified description details use of the skin care device 10 at an umbilical cord site, it should be appreciated that the device 10 may be used in other locations where sensitive skin needs to be protected, for example, from dehydration. For example, the skin care device 10 may be sized as necessary for use on the back of the infant 14 separate from, or in additional to, the abdomen 16.

Additionally, the illustrative skin care device 10 could also be used in other applications such as with gastric tubes and other surgical implants, particularly with those patients having sensitive skin.

Although the invention has been described in detail with reference to certain preferred embodiments, variations and modifications exist within the spirit and scope of the invention as described and defined in the following claims.

1. A skin care device for an infant comprising:
a base including an upper surface, a lower surface and a center opening configured to receive an umbilical cord of the infant, the lower surface having a silicone adhesive;
wherein the silicone adhesive is secured to an outer epidermis layer of the infant, the outer epidermis layer coupled to an inner dermis layer of the infant, the adhesive tension between the silicone adhesive and the outer epidermis layer being less than the adhesive tension between the outer epidermis layer and the inner dermis layer;
a liner removably coupled to the silicone adhesive; and
a upper layer coupled to the upper surface of the lower layer.

2. The skin care device of claim 1, wherein the lower layer includes a non-woven fabric supporting the silicone adhesive.

3. The skin care device of claim 1, wherein the lower layer is fenestrated.

4. The skin care device of claim 3, wherein the lower layer includes a transparent window.

5. The skin care device of claim 1, wherein a temperature probe is supported by the lower layer.

6. The skin care device of claim 1, wherein an ECG probe is supported by the lower layer.

7. The skin care device of claim 1, wherein a positioning slot extends within the lower layer outwardly from the center opening.

8. The skin care device of claim 1, wherein the upper layer includes an upwardly extending first securement tab and an upwardly extending second securement tab positioned on opposing sides of the center opening.

9. The skin care device of claim 8, wherein at least one of the first securement tab and the second securement tab includes an adhesive facing the other of the second securement tab and the first securement tab, the first securement tab being coupled to the second securement tab to define a bridge extending over the center opening and capturing an umbilical line between the first securement tab and the second securement tab.

10. The skin care device of claim 1, wherein the lower layer includes a first antimicrobial, and at least one of the first securement tab and the second securement tab includes a second antimicrobial.

11. The skin care device of claim 10, wherein the first antimicrobial comprises dialkylcarbamoyl chloride (DACC), and the second antimicrobial comprises chlorhexidine (CHG).

12. The skin care device of claim 1, wherein the lower layer is semi-transparent for phototherapy transmissions up to 490 nm.

13. The skin care device of claim 1, wherein the lower layer provides a water vapor transmission rate of at least 2000 g/m² per 24 hours.

14. The skin care device of claim 13, wherein the lower layer provides a water vapor transmission rate of between 2000 g/m² per 24 hours and 3800 g/m² per 24 hours.

15. A skin care device for an infant comprising:
a lower layer including an upper surface, a lower surface, a center opening configured to receive an umbilical cord of the infant, and a positioning slot extending outwardly from the center opening, the lower surface having an adhesive;
an upwardly extending first securement tab and an upwardly extending second securement tab positioned on opposing sides of the center opening; and
wherein the lower layer includes a first antimicrobial, and at least one of the first securement tab and the second securement tab includes a second antimicrobial.

16. The skin care device of claim 15, wherein the lower layer includes a non-woven fabric supporting the adhesive.

17. The skin care device of claim 15, wherein the lower layer includes a sensor opening, and a temperature probe received within the sensor opening.

18. The skin care device of claim 15, wherein at least one of the first securement tab and the second securement tab includes an adhesive facing the other of the second securement tab and the first securement tab, wherein the first securement tab is coupled to the second securement tab to define a first bridge extending over the center opening and capturing a first umbilical line between the first securement tab and the second securement tab.

19. The skin care device of claim 18, further comprising:
an upwardly extending third securement tab and an upwardly extending fourth securement tab positioned on opposing sides of the center opening; and
wherein at least one of the third securement tab and the fourth securement tab includes an adhesive facing the other of the fourth securement tab and the third securement tab, wherein the third securement tab is coupled to the fourth securement tab to define a second bridge extending over the center opening and capturing a second umbilical line between the third securement tab and the fourth securement tab.

20. The skin care device of claim 15, wherein the first antimicrobial comprises dialkylcarbamoyl chloride (DACC), and the second antimicrobial comprises chlorhexidine (CHG).

21. The skin care device of claim 15, wherein the lower layer is semi-transparent for phototherapy transmissions up to 490 nm.

22. The skin care device of claim 15, wherein the lower layer provides a water vapor transmission rate of at least 2000 g/m² per 24 hours.

23. The skin care device of claim 22, wherein the lower layer provides a water vapor transmission rate of between 2000 g/m² per 24 hours and 3800 g/m² per 24 hours.

24. The skin care device of claim 15, wherein the adhesive is secured to an outer epidermis layer of the infant, the outer epidermis layer coupled to an inner dermis layer of the infant, the adhesive tension between the adhesive and the outer epidermis layer being less than the adhesive tension between the outer epidermis layer base and the inner dermis layer.

25. A skin care device for an infant comprising:
a lower layer including an upper surface, a lower surface and a center opening configured to receive an umbilical cord of the infant, the lower surface having an adhesive;
wherein a positioning slot extends within the lower layer outwardly from the center opening;
wherein the lower layer includes a sensor opening receiving a temperature probe;
a liner removably coupled to the adhesive of the lower surface;
an upwardly extending first securement tab and an upwardly extending second securement tab positioned on opposing sides of the center opening;
wherein at least one of the first securement tab and the second securement tab includes an adhesive facing the other of the second securement tab and the first securement tab, wherein the first securement tab is coupled to the second securement tab to define a first bridge extending over the center opening and capturing a first umbilical line between the first securement tab and the second securement tab;
wherein the lower layer is semi-transparent for phototherapy transmissions of at least 490 nm; and
wherein the lower layer provides a water vapor transmission rate of at least 2000 g/m² per 24 hours.

26. The skin care device of claim 25, wherein the lower layer includes a first antimicrobial, and at least one of the first securement tab and the second securement tab includes a second antimicrobial.

27. The skin care device of claim 26, wherein the first antimicrobial comprises dialkylcarbamoyl chloride (DACC), and the second antimicrobial comprises chlorhexidine (CHG).

28. The skin care device of claim 25, further comprising:
an upwardly extending third securement tab and an upwardly extending fourth securement tab positioned on opposing sides of the center opening; and
wherein at least one of the third securement tab and the fourth securement tab includes an adhesive facing the other of the fourth securement tab and the third securement tab, wherein the third securement tab is coupled to the fourth securement tab to define a second bridge extending over the center opening and capturing a second umbilical line between the third securement tab and the fourth securement tab.

29. The skin care device of claim 25, wherein the lower layer provides a water vapor transmission rate of between 2000 g/m² per 24 hours and 3800 g/m² per 24 hours.

30. The skin care device of claim 25, wherein the silicone adhesive is secured to an outer epidermis layer of the infant, the outer epidermis layer coupled to an inner dermis layer of the infant, the adhesive tension between the silicone adhesive and the outer epidermis layer being less than the adhesive tension between the outer epidermis layer base and the inner dermis layer;
